(19) United States
(12) Patent Application Publication
(10) Pub. No.: US 2005/0042174 A1
(43) Pub. Date: Feb. 24, 2005

(54) Combined doses

(75) Inventors: Thomas Nilsson, Mariefred (SE); Mattias Myrman, Stockholm (SE); Claes Friberg, Akers Styckebruk (SE); Sven Calander, Strangnas (SE)

Correspondence Address: OBLON, SPIVAK, MCCLELLAND, MAIER & NEUSTADT, P.C., 1940 DUKE STREET, ALEXANDRIA, VA, 22314, US

(73) Assignee: Microdrug AG, Hergiswil NW (CH)

(21) Appl. No.: 10/870,907

(22) Filed: Jun. 21, 2004

Related U.S. Application Data
(60) Provisional application No. 60/500,262, filed on Sep. 5, 2003.

(30) Foreign Application Priority Data
Jun. 19, 2003 (SE) 0301816-5
Nov. 17, 2003 (SE) 0303050-9

(51) Int. Cl. A61L 9/04; A61K 9/14

(52) U.S. Cl. 424/46

(57) ABSTRACT

A method and pharmaceutical dry powder combination of doses for the prophylaxis or treatment of a respiratory disorder in a user by inhalation of metered dry powder combinations of doses of finely divided dry medication powders. Medicaments for forming a combination of doses are selected from a group of beta2-agonists, a group of anticholinergic agents and a group of anti-inflammatory corticosteroids. Metered dry powder medicinal combinations of doses comprising separately metered deposits of medicinally suitable quantities of each of the selected medicaments are prepared. The medicinal combination of doses are introduced into an adapted inhaler device for a generally simultaneous or sequential delivery during the course of a single inhalation by a user, such that each one of the delivered medicinal combination of doses is composed of a high proportion of de-aggregated fine particles of the selected medicament or medicaments.

### TECHNICAL FIELD

The present invention relates to combinations of doses of certain asthma medicaments for administration by an oral inhalation route to a user in need of treatment of asthma or other respiratory disorders. In particular, a combination of doses of a beta2-agonist, an anticholinergic agent and an anti-inflammatory steroid are packaged to suit a new method of aerosolizing a selected individual combination of doses into air and more particularly, the invention relates to combinations of separate dry powder entities of said asthma medicaments constituting combinations of doses intended for delivery in a single inhalation by a user.

### BACKGROUND

Asthma and chronic obstructive pulmonary disease (COPD) affect more than 30 million people in the United States. More than 100,000 deaths each year are attributable to these conditions. Obstruction to airflow through the lungs is the characteristic feature in each of these airway diseases, and the medications utilized in treatment are often similar.

Up to 5% of the US population suffers from asthma, a respiratory condition characterized by airway inflammation, airway obstruction (at least partially reversible), and airway hyperresponsiveness to such stimuli as environmental allergens, viral respiratory-tract infections, irritants, drugs, food additives, exercise, and cold air. The major underlying pathology in asthma is airway inflammation. Inflammatory cell—eosinophils, CD4+ lymphocytes, macrophages, and mast cells—release a broad range of mediators, including interleukins, leukotrienes, histamine, granulocyte-colony-stimulating factor, and platelet aggregating factor. These mediators are responsible for the bronchial hyperreactivity, bronchoconstriction, mucus secretion, and sloughing of endothelial cells.

Chronic obstructive pulmonary disease (COPD) is a widespread chronic lung disorder encompassing chronic bronchitis and emphysema. The causes of COPD are not fully understood. Experience shows that the most important cause of chronic bronchitis and emphysema is cigarette smoking. Air pollution and occupational exposures may also play a role, especially when combined with cigarette smoking. Heredity also causes some emphysema cases, due to alpha1 antitrypsin deficiency.

Chronic bronchitis is caused by excess mucus production in the lungs causing infection, which in turn causes inflammation and swelling, thus narrowing the bronchial tubes. This narrowing impedes airflow in and out of the lungs, causing shortness of breath. The condition usually begins with intermittent tracheobronchitis; however, repeated attacks occur until the disease and its symptoms persist continuously. If left untreated or if the patient continues to smoke, chronic bronchitis can lead to emphysema.

Administration of asthma drugs by an oral inhalation route is very much in focus today, because of advantages offered like rapid and predictable onset of action, cost effectiveness and high level of comfort for the user. Dry powder inhalers (DPI) are especially interesting as an administration tool, compared to other inhalers, because of the flexibility they offer in terms of nominal dose range, i.e. the amount of active substance that can be administered in a single inhalation. So far most development efforts have been directed towards producing effective drugs and formulations for specific abnormal conditions and not so much towards developing combined dose metering, forming methods and a suitable delivery device, i.e. the inhaler.

When inhaling a combined dose of dry medication powder it is important to obtain by mass a high fine particle fraction (FPF) of particles with an aerodynamic size preferably less than 5 μm in the inspiration air. The majority of larger particles does not follow the stream of air into the many bifurcations of the airways, but get stuck in the throat and upper airways. It is not uncommon for prior art inhalers to have an efficacy of 10-20% only, i.e. only 10-20% of the metered dose by mass is actually delivered as particles with an aerodynamic size less than 5 μm. Since most drugs may have undesirable side effects, e.g. steroids delivered to the system, it is important to keep the dosage to the user as exact as possible and to design the delivery system, e.g. an inhaler, such that the efficacy becomes much higher than 10-20%, thereby reducing the requited amount of drug in the dose. Common, serious adverse effects of corticosteroids are osteoporosis, growth retardation, candidiasis and muscle injuries. Common, serious adverse effects of beta2-agonists are tremor, palpitations, headache, dizziness and oropharyngeal irritation.

In search of methods and devices for improving dose efficacy and reducing the dosages necessary for adequate control of symptoms and respiratory disorders, some developments are to be noted. For instance, in an article in Journal of Aerosol Medicine, Volume 12, Supplement 1, 1999, Pp. S-33-S-39 the authors Pavia and Moonen report clinical studies comparing therapy efficacy of a “soft mist inhaler” Respimat from Boehringer Ingelheim KG with that of a metered dose inhaler (MDI). The studies show that the Respimat gives at least the same therapeutic bronchodilating effect as the MDI but using only half or less of the dosage in the MDI. The Respimat produces a slow-moving cloud of medicament droplets with a high fine particle fraction in a prolonged dose delivery taking in the order of one second, which reduces the deposition in the oropharynx and raises the topical delivery to the correct site of action in the lung. The challenge of developing inhalers capable of producing a delivered dose with a high fine particle fraction in a prolonged dose delivery is discussed in another article in Journal of Aerosol Medicine, Volume 12, Supplement 1, 1999, Pp. S-3-S-8 by the author Ganderton.

Interestingly, research during the past decade into respiratory diseases, their prophylaxis and treatment, has shown conclusively that simultaneous administration of combinations of different medicaments may improve the clinical condition of patients considerably. See for instance National Heart, Lung, and Blood Institute “Guidelines for the Diagnosis and Management of Asthma” NIH Publication No. 97-4051 July 1997, where a combined use of a long-acting beta2-agonist and a corticosteroid drug is recommended, formoterol and fluticasone being mentioned as good examples of substances of the respective groups. The document stresses that it is important to reduce the adverse effects of the medicaments in general and inhaled corticosteroids in particular by reducing to a minimum the dosage, which still keeps the inflammation under control. At the time when these guidelines were compiled no medical products were available offering comprehensive combined medication together with suitable administration tools, at least not to the American public. The only possibility at the time was to combine by prescribing different medicaments, preferably for inhalation, one from each group and separate inhalers for administration. This method of treatment was well known to physicians at the time. Several studies in the mid-1990's have shown that by adopting a combined treatment it has been possible to reduce the dose of steroid compared to using the steroid as background treatment and a beta2-agonist as rescue medicine, besides improving lung function and reducing severity and frequency of attacks of dyspnoea.

For instance, in Switzerland patients diagnosed with asthma have been prescribed FORADIL (formoterol, a bronchodilating substance) together with PULMICORT (budesonide, an anti-inflammatory steroid) since the 1980's for treatment of their asthma. Until recently, however, different asthma medicaments have generally been administered separately, in sequence or by separate routes, not in compositions comprising more than one active ingredient. However, there are several published patent applications and approved patents teaching methods of treating respiratory disorders like asthma and chronic obstructive pulmonary disease (COPD) as well as pharmacologic compositions of different biological and chemical substances for this purpose, where the combinations offer overall advantages in the treatment of these conditions. See for instance EP 0416950B1 “Medicaments”, EP 0416951B1 “Medicaments comprising salmeterol and fluticasone”, EP 0613371B1“New combination of formoterol and budesonid”, WO 98/15280 “New combination”, WO 00/48587 “Combinations of formoterol and fluticasone propionate for asthma”, WO 01/70198A1 “Stabilized dry powder formulations”, WO 01/78737A1 “Medical combinations comprising formoterol and budesonide”, WO 01/78745A1 “Medical combinations comprising formoterol and fluticasone propionate”, WO 02/28368A1 “New combination for the treatment of asthma”, WO 03/013547A1 “Pharmaceutical composition comprising salmeterol and budesonid for the treatment of respiratory disorders”, U.S. Pat. No. 5,603,918 “Aerosol composition of a salt of ipratropium and a salt of albuterol”, U.S. Pat. No. 6,433,027 “Medicament compositions based on tiotropium bromide and formoterol fumarate”, US 2003/0096834 “Pharmaceutical compositions”, WO 00/47200 “Combinations of formoterol and a tiotropium salt”. However, the quoted documents deal with aspects of formulating, processing, stabilizing and using mixtures of at least two ingredients. The mixing ratios between active ingredients and compositions thereof including suitable carriers, solvents and excipients are generally focused upon, not methods of administration or devices for that purpose.

A further document WO 01/78735, Sanders et al., claims a method of treating a respiratory disorder by administering an effective amount of the active ingredients formoterol and fluticasone separately, sequentially or simultaneously, provided that the ingredients comprise separate compositions. A dry powder inhaler containing formoterol and fluticasone in separate compositions is also claimed. However, Sanders et al. fail to teach how the skilled person should perform the method. Sanders goes on to teach that each of the active ingredients should be administered as separate compositions preferably once or twice daily. The document discloses that the claimed method may comprise an improvement of from 35-50% (in glucocorticoid receptor translocation into the nucleus) over known combination therapies, but no relevant information is given as to why the claimed method is superior and novel in relation to prior art, e.g. as exemplified in the previously mentioned documents. Further, no distinctive characteristics of the dry powder inhaler are disclosed, separating the inhaler from prior art inhalers.

A common denominator for the quoted documents is that they have as their first objective to simplify and improve asthma therapy for the user. A simpler, twice daily administration by inhalation of well-known, well-documented medicaments, one of which selected to address symptoms of bronchoconstriction and the other to address an underlying inflammation of the bronchi, has proved in clinical testing to result in high user acceptance and compliance with a prescribed dosing regimen. The results of this therapy are in many reports compared with therapy using only the one or the other medicament, sometimes with increased dosages, or compared to separate prescriptions of said medicaments, but without specific instructions to the user on how to combine the administration of the two medicaments to achieve the best effect.

It comes as no surprise to a person of ordinary skill in the art that combining two well-documented medicaments, one to give quick relief of symptoms and the other to treat the cause in the long term, would be a good idea. The quoted documents all teach compositions of a beta2-agonist, preferably a long-acting bronchodilating drug with fast onset like formoterol, and either a corticosteroid, i.e. an anti-inflammatory drug e.g. budesonide or fluticasone propionate, or an anticholinergic agent, e.g. ipratropium bromide or tiotropium bromide, in mixtures using effective amounts of the drugs and varying ratios between drugs depending on the condition, age, sex etc of the patient. The disclosed inventions in the quoted documents rely on existing MDI or DPI inhalers to do the job of delivering the medicament mixtures using a single inhaler. The documents also teach various techniques of combining two drugs in order to simplify self-therapy for asthmatics. The disclosed techniques range from mixing the drugs in various ways into an indivisable medicament to supplying medical kits composed of separately packaged doses for insertion in separate inhalers for separate, sequential delivery of the selected drugs. In the latter case it is difficult to see where the improvement for the user is lying.

None of the quoted documents indicate that the claimed medicament composition offers a therapeutic benefit, or quote clinical studies in support of such benefits, in comparison with separate, sequential delivery of the equivalent active medicaments. On the contrary, several documents teach that there is no therapeutic difference between delivering the active medicaments substantially simultaneously, sequentially or separately.

Furthermore, none of the quoted documents discusses in depth the importance of formulating a dry powder medicament for inhalation, e.g. the claimed compositions, such that an optimum distribution of particle aerodynamic diameters for optimum therapeutic effects from the selected drugs are arrived at. Also, there is no general recommendation as to an order in which the different medicament doses, if physically separated, should be delivered to an inhaling user, presumably because a concept of delivering, in a single inhalation, a combination of doses composed of separate, individual doses of each medicament is unusual, if not unknown, in prior art. Likewise, a concept of cutting back the quantities of active ingredients in the combined doses by implementing a giant increase in efficacy in the delivered dosage by adopting a prolonged dose delivery is also practically unknown in prior art.

The preferred embodiment of the inventions of the quoted documents is a mixture of the active drugs involving preferred prior art methods of preparing combined doses by razing the ingredients. It is, however, difficult to mix dry medicament powders and optional excipients in a certain proportion consistently. The proportions in such a metered combined dose cannot easily be controlled, because the ratio of medicaments in an individual, combined dose depends significantly on the particle forces existing in each medicament powder, between particles of different medicaments and between medicament powders and dose packaging materials. Hence, actual variations in the ratio between active ingredients from combined dose to combined dose may be too large, causing serious problems if a potent ingredient is delivered in a higher or lower amount than expected.

Bronchodilating medicaments such as short-acting beta2-agonists have been used for many years in control of asthma and particularly as rescue medicines, administered as needed Salbutamol, for instance, has very fast onset but short duration and may be administered, preferably by inhalation, several times per day in order to control attacks of dyspnoea, such that a puff of the drug provides immediate relief. Salmeterol and formoterol, both long-acting beta2-agonists, are bronchodilators, which have been used with great success for more than 20 years in the treatment of asthma. Different enantiomers of e.g. formoterol exist, RR, SS, SR and RS with rather different efficacies as bronchodilators. Thus, the recommended dosage of formoterol must be adjusted depending on which enantiomers are present and in what ratios in any particular formulation of formoterol. Formoterol is preferred over salmeterol by many asthmatics because it has a fast onset. Formoterol, but not salmeterol may be used as a rescue medicine for a quick relief of symptoms during an asthma attack. However, none of the beta2-agonists, have any significant effect on underlying inflammation of the bronchi. Besides the already wellknown adverse side effects of long-acting beta2-agonists, a recent study in the US reports statistically positive evidence that salmeterol may be at the root of premature deaths caused by an acute asthma attack among salmeterol users with respiratory disorders. This is especially pronounced in the afro-american population, which has induced FDA to issue warning messages to users. Apparently, no evidence points in this very disturbing direction for short-acting or for other long-acting beta2-agonists.

Anticholinergic agents, e.g. ipratropium, oxitropium and tiotropium, particularly ipratropium bromide and tiotropium bromide, are also effective bronchodilators, but act in a different way to beta2-agonists, with relatively fast onset and long duration of action, especially ipratropium and tiotropium, of which the latter may be active for up to 24 hours. Adverse side effects for inhaled anticholinergic agents are insignificant, dryness of mouth and constipation are most common.

Steroids, e.g. budesonide, fluticasone, beclomethasone, on the other hand, are anti-inflammatory substances, which during the past two decades have proved to be very successful and potent drugs, fluticasone and budesonide in particular, in reducing inflammation of nasal passages and bronchial tissue to make breathing easier. However, anti-inflammatory steroids do not offer immediate relief for a person suffering an asthma attack, but the drug will help to manage the inflammation and reduce the severity and number of exacerbations, if taken regularly. Generally, although adverse side effects differ slightly from steroid to steroid, side effects may be quite serious and it is therefore imperative to keep the dosage of steroids to a minimum and direct the medicament topically to the site of action in the lung, with smallest possible systemic effect.

National health-care institutions in most countries have been slow to actively promote the use of combined therapy, in the early days because of exaggerated fear, as it turned out, of negative long-term side effects from the beta2-agonist, although in the last decade combined treatment has been listed as an open option for physicians in treating asthma patients. Thus, the full potential has not been realized of the obvious advantages, which may be achieved in a physician-controlled therapy using a combination of bronchodilators and an ant-inflammatory drug in management of asthma and COPD. A reason for the slowness has been a lack of understanding among researchers and scientists of the complex mechanisms of airways diseases. Today, although much remains to be learned about asthma and COPD, many clinical tests have shown conclusively that combination therapy is working and provides good therapeutic results for many asthmatics.

Because it is very difficult to diagnose a respiratory disorder in a person correctly, i.e. the degree of partial chronic obstructive pulmonary disease, inflammatory status and degree of allergetic asthma, it is difficult to know exactly what therapy is the best for the patient. Prescribing a combined treatment using a medicinal combination of a beta2-agonist, preferably with fast onset, an anticholinergic agent and an anti-inflammatory steroid offers important advantages:
a the beta2-agonist and the anticholinergic agent have both a bronchodilating effect but in different ways, giving a synergetic, fast relief and a more or less long-term effect
the steroid will reduce the underlying inflammation, giving a better long-term control of the disorder
each medicament dosage in the combination may be significantly reduced, compared to treatment using only one medicament, thus reducing unwanted side effects
easy self-administration by users is possible
improved user confidence and compliance

Thus, there is a need for improvements regarding methods of treating respiratory disorders using a combination of consistently metered doses of bronchodilators and anti-inflammatory steroids for co-ordinated administration by inhalation using a new type of inhaler device.

### SUMMARY

The present invention discloses a method for the administration by inhalation of a co-ordinated combination of metered doses of finely divided dry powders of two different bronchodilators and optionally an anti-inflammatory steroid by means of an adapted inhaler designed for a prolonged delivery of the combination of doses. A combination of metered dry powder medicinal doses A′, B′ and C′ is prepared comprising separately metered deposits of a beta2-agonist (A), e.g. salbutamol, formoterol or salmeterol, and an anticholinergic agent (B), e.g. tiotropium, ipratropium or oxitropium, and optionally an anti-inflammatory steroid (C), e.g. fluticasone or budesonide. Medicaments A, B and C respectively may include pharmaceutically acceptable salts, enantiomers, racemates, hydrates, solvates or mixtures thereof, in suitable quantities and ratios, and may optionally include diluents or other excipients. “Salbutamol”, “formoterol” or “salmeterol” refer hereinafter to all the various chemical forms of the active substances, which are suitable for an intended therapeutic effect, e.g. salbutamol sulphate, formoterol fumarate and salmeterol xinafoate. “Oxitropium”, “ipratropium” and “tiotropium” refer hereinafter to all the various chemical forms of the active substance, which are suitable for an intended therapeutic effect and in particular to a bromide salt of the respective substances. “Budesonide” and “fluticasone” refer hereinafter to all the various chemical forms of the active substance, which are suitable for an intended therapeutic effect, and in particular to fluticasone propionate. Because of the potency of the respective drugs it may be necessary to dilute the active substances separately using a pharmacologically acceptable diluent or excipient in order to secure the correct amounts as well as the ratio between the active substances in the formed combination of doses. Careful metering of the entities of deposited powder constituting the combination of doses, A′, B′ and C′ respectively, may control the very small, individual quantities of active substances tightly. Hence, the sum of the metered entities is the sum of the doses A′+B′+C′, which constitutes the metered quantities of powder of the combination of doses.

Different dosing regimens may be chosen for different patients based on what treatment is considered best for an individual patient as user of an inhaler device adapted for a prolonged delivery of doses. In a first preferred embodiment each combination of doses comprises metered doses A′, B′ and C′ in medicinally effective quantities and ratios of the respective three medicaments A, B and C. Each such combination of doses is coordinated on a common dose bed arranged for a prolonged delivery in a single inhalation by the user. The number of such administrations per day may range typically from one to four or five according to a prescribed dosing regimen.

In a second preferred embodiment, different dose combinations may be used in an inhaler device adapted for a prolonged delivery of doses. For instance, a combination of doses comprising only two bronchodilating medicaments, A and B, may be administered typically twice or three times a day, but interspersed with administration of a combination of doses comprising all three medicaments, A, B and C, typically once or twice per day according to a selected prescribed dosing regimen.

In a third preferred embodiment, similar to the second embodiment, a combination of doses comprising only two bronchodilating medicaments, A and B, may be administered typically twice or three times a day using the inhaler for prolonged delivery. But administration of the combination of A′+B′ may be interspersed with administration of a dose comprising only medicament C, typically once or twice per day using the same inhaler device according to a selected prescribed dosing regimen.

In yet another preferred embodiment, different dose combinations may be administered using different inhalers, where the dose combinations and inhalers constitute a medicinal kit for use in the treatment of asthma, COPD and other respiratory disorders.

A user introduces a medicinal combination of doses comprising the separated powder entities of bronchodilators and optional steroid into arm adapted inhaler device for a prolonged delivery of the combination of doses during the course of a single inhalation. A prolonged delivery of the separated entities of powder deposits of the respective medicaments is preferably arranged to be sequential and more preferably arranged such that the beta2-agonist (A) is delivered first, the anticholinergic agent (B) immediately thereafter optionally followed by the anti-inflammatory steroid (C) last, so that e.g. fast-acting salbutamol and ipratropium may reach deeper into the lung for topical action and fast onset, while optional budesonide or fluticasone may be topically deposited more in the central lung area for best effect with as little systemic effect as possible. The delivered doses are composed of a high proportion of de-aggregated fine particles of the selected medicaments respectively, and the particle flows are preferably separated in time, whereby an intended prophylactic, therapeutic and psycologic effect on the user is achieved. The ease of use, the immediate relief of symptoms during asthma attacks and the successful long-term control of the asthmatic condition ensures a high level of compliance from patients with the typically one to four times daily dosing regimen using the presented combination of doses.

Furthermore, combinations of pharmaceutical dry powder doses are disclosed. The doses are adapted for inhalation, for the prophylaxis or treatment of a respiratory disorder. The combinations of pharmaceutical dry powder doses A′, B′ and optionally C′ are prepared comprising separate entities of metered deposits of medicinally effective quantities of medicaments A, B and C respectively, optionally including diluents or excipients, where the sum of the entities constitutes the metered quantities of powder in the pharmaceutical combination of doses arranged for introduction into an inhaler device adapted for a prolonged dose delivery.

The present method is set forth by the independent claim 1, and 9 and the dependent claims 2-8 and 10-16, and pharmaceutical combinations of doses are set forth by the independent claim 17 and the dependent claims 18 to 23. A use is set forth by independent claim 24 and a medical kit is set forth by the independent claim 25 and the dependent claims 26 and 27.

### BRIEF DESCRIPTION OF THE DRAWINGS

The invention, together with further objects and advantages thereof, may best be understood by referring to the following detailed description taken together with the accompanying drawings, in which:

FIG. 1 illustrates in top and side views a first embodiment of combinations of doses comprising two medicament entities deposited in separate compartments onto a doses bed;

FIG. 2 illustrates in top and side views a second embodiment of combination of doses comprising three medicament entities deposited in separate compartments onto a dose bed;

FIG. 3 illustrates in top and side views a third embodiment of combination of doses comprising two parallel medicament entities deposited onto a dose bed;

FIG. 4 illustrates in top and side views a fourth embodiment of combination of doses comprising several medicament entities and separating excipient entities deposited onto a dose bed;

FIG. 5 illustrates in top and side views a fifth embodiment of combination of doses comprising four medicament entities and separating excipient entities deposited onto a dose bed;

FIG. 6 illustrates in top and side views a sixth embodiment of combination of doses comprising two parallel medicament entities deposited on top of one another onto a dose bed;

FIG. 7 illustrates in top and side views a seventh embodiment of a combination of doses comprising two medicament entities deposited on top of one another onto a dose bed, but separated by a deposited excipient entity;

FIG. 8 illustrates in top and side views another embodiment of a combination of doses comprising two medicament entities separately deposited onto a dose bed;

FIG. 9 illustrates in top and side views yet another embodiment of a combination of doses comprising two medicament entities separately deposited onto a dose bed, but with some degree of overlap;

FIG. 10a illustrates in a sectional view an example of a combination of doses comprising two medicament entities deposited on top of one another but separated by a deposited excipient entity onto a dose bed and adjacent to the combination of doses a nozzle in a starting position before the combination of doses are released;

FIG. 10b illustrates in a sectional view an example of a combination of doses comprising two medicament entities deposited on top of one another but separated by a deposited excipient entity onto a dose bed and adjacent to the combination of doses a nozzle in a relative motion sucking up the powder particles to be dispersed into the air stream;

FIG. 11 illustrates in top and side views an embodiment of a combination of doses comprising three different medicaments, intended for a sequential delivery, deposited in three entities in separate compartments onto a dose bed;

FIG. 12 illustrates in top and side views another embodiment of a combination of doses, intended for a mixed simultaneous and sequential delivery, comprising three medicaments deposited in several entities onto separate areas of a common dose bed;

FIG. 13 illustrates in top and side views a third embodiment of a combination of doses, intended for a sequential delivery, comprising three medicaments deposited in several entities with separating excipient entities deposited onto a common dose bed.

### DETAILED DESCRIPTION

The present invention discloses a new combination of active asthma drugs comprising a coordinated combination of metered doses of two or optionally three medicaments: (A) a beta2-agonist, (B) an anticholinergic agent and (C) a steroid, where (A) is represented by salbutamol, particularly salbutamol sulphate, or salmeterol, particularly salmeterol xinafoate, or formoterol, particularly formoterol fumarate, and (B) is represented by oxitropium, or preferably ipratropium or more preferably tiotropium, all of which advantageously in bromide form and (C) is represented by budesonide or preferably fluticasone, particularly fluticasone propionate. In a further aspect, the invention discloses a new therapeutic method of treating respiratory diseases like asthma by delivering such co-ordinated combinations of doses by an inhalation route, to a user of a dry powder inhaler [DPI]. “Asthma” is used in this document as a generic term for the different respiratory disorders known in the field of medicine.

In the context of this application the word medicament is defined as a pharmacologic substance, which comprises at least one chemically or biologically active agent. Further, a medicament may exist in a pure form of one or more pure active agents, or a medicament may be a compound comprising one or more active agents, optionally formulated together with other substances, e.g. enhancers, carriers, diluents or exipients. Hereinafter, the term “excipient” is used to describe any chemical or biologic substance mixed in with a pure active agent for whatever purpose. In this document, only medicaments in dry powder form are discussed. Substances A, B and C respectively include pharmaceutically acceptable salts, enantiomers, racemates, hydrates, solvates or mixtures thereof, in effective quantifies and ratios, and may optionally include diluents or other excipients.

A “dose bed” is henceforth defied as a member capable of harboring “a co-ordinated combination of metered doses comprising at least two entities of dry powders” in the document referred to as “a combination of doses”. The term “combinations of doses” refers to different combinations of metered doses of different medicaments selected among the groups A, B and C and where the absolute and relative quantities of medicaments in the respective doses constitute a particular combination of doses. Thus, a nominal change in a dose quantity defines a new combination, although it is otherwise identical to previous combinations. All combinations of doses are intended for delivery, one combination at a time, to a user of a DPI in a single inhalation performed by the user. Different types of pharmaceutical blister packs or capsules are included in the term “dose bed”. In the present invention a combination of doses for treating asthma comprises metered, deposited entities of two bronchdilators and optionally an anti-inflammatory steroid, the mentioned medicaments optionally including excipients. The dose bed may be divided in two or more areas or incorporate two or more compartments, i.e. cavities of suitable volume, intended for deposited entities of dry powders of the selected medicaments. In a preferred embodiment the combination of doses is packaged for a prolonged delivery, i.e. the delivery period for the combination of doses is in a range from 0.01 to 6 s, usually in a range from 0.1 to 2 seconds, delivery taking place sometime during the course of an inhalation as controlled by a purposefully designed DPI, adapted for administration of combinations of doses. Advantageously, such a DPI adopts an Air-razor method of gradual aerosolization of the combination of doses by introducing a relative motion between an air-sucking nozzle and the powder doses. Advantages of a prolonged delivery of a dose for inhalation are disclosed in our U.S. Pat. No. 6,571,793 B1 (WO 02/24264 A1), which is hereby incorporated in this document in its entirety as a reference.

A preferred embodiment of a combination of doses use a dose bed split up in two or optionally three separate compartments, where each compartment is intended for a metered deposition of a particular asthma medicament, in this case a beta2-agonist A, an anticholinergic agent B and a steroid C, where A is represented by salbutamol, particularly salbutamol sulphate, or salmeterol, particularly salmeterol xinafoate or formoterol, particularly formoterol fumarate, and B is represented by oxitropium, or preferably ipratropium or more preferably tiotropium, all of which advantageously in bromide form and C is represented by budesonide or preferably fluticasone, particularly fluticasone propionate. Each compartment containing at least one metered entity of one and the same medicament powder may then be sealed, e.g. by foiling, such that the different medicaments in the different compartments of the dose bed cannot interact in any way and cannot be contaminated by foreign substances or moisture. Alternatively, a common foil may enclose all compartments, and sealing between compartments may be excluded if individual sealing is not a GMP or medicinal requirement. A dose carrier is normally engaged to carry at least one dose bed loaded with a combination of doses, whereby the dose carrier may be inserted into a DPI for administering one or more combinations of doses, which may be the same or different, to a user in need of treatment. A suitable carrier of dose beds loaded with combinations of doses is disclosed in our American patent publication U.S. Pat. No. 6,622,723 B1 (WO 01/34233 A1), which is hereby incorporated in this document in its entirety as a reference. However, a dose bed may be designed to act as a carrier, intended for direct insertion into a DPI. A suitable DPI for a continuous dose delivery is disclosed in our U.S. Pat. No. 6,422,236 B1, which is hereby incorporated in this document in its entirety as a reference.

If complete physical separation of the deposited entities of the three medicaments making up the combination of doses is not required but some degree of overlap or mixing is acceptable from a physical, chemical and medical point of view, then other methods of separating the deposited entities may be implemented. Depending on what degree of mixing is permitted or in some cases even desired, different ways of separating medicament entities must be adopted. For example, the dose bed may use separate indentations where different powders should be deposited, but flat target areas for separate deposits in a single plane on the dose bed are equally possible. In another embodiment the three medicaments are deposited sequentially dot-wise or string-wise onto three target areas of the dose bed. If necessary, to stop chemical or biological interaction or decomposition caused by, for example, adjacent medicament powders being incompatible, an isolating, biologically acceptable, inert substance like carbohydrates, e.g. glucose or, lactose, may be deposited between the medicament entities. When the combined dose entities have been completely formed they are usually sealed from ingress of dirt and moisture by a foil covering the entire dose bed. A method of depositing microgram and milligram quantities of dry powders using electric field technology is disclosed in our U.S. Pat. No. 6,592,930 B2, which is hereby incorporated in this document in its entirety as a reference.

Forming a combination of doses comprising two or optionally three medicaments of separate dry powder formulations may be done in different ways, known in prior art. The invention discloses that the finely divided powders to be included in the combination of doses, e.g. salbutamol, ipratropium and fluticasone respectively, need not be mixed or processed together prior to dose forming and, indeed, should be kept separated during dose forming as well as after the respective entities of the combination of doses have been formed and sealed. The medicament entities constituting a combination of doses are thus kept separated on the common dose bed by suitable methods, as described in the foregoing, until a selected combination of doses is about to be delivered by an inhalation route to a user, whereby the included medicament entities are delivered, preferably in sequence, separated in time and therefore not intermixed in the inhaled air leaving the mouthpiece of the DPI.

The present invention offers inherent manufacturing advantages in comparison with prior art methods, which are based on mixing the active ingredients in bulk quantities, generally including diluents and/or carriers before forming doses. The consequence of this mixing step in the manufacturing process, apart from the regulatory problem of proving the mixture as such, is that many different blends of mixture must be made and verified to provide the correct ratios between the active ingredients in order to correspond to given therapeutic requirements, since different patients need different ratios, besides correct quantities. Disregarding the problem of verifying a mixture in bulk quantity, besides the problem of verifying the actual ratio between ingredients in each individual dose, a further consequence of the mixing step is the extra time required for producing, storing and verifying the mixture before and during the dose forming process. Also to be considered is the circumstance that it is not uncommon for active substances to have a limited period of stability, which is often even shorter when mixed with other active ingredients.

The present invention avoids all of these problems, since the active ingredients are kept separate, optionally in a mixture with excipient(s), all the way through the dose manufacturing process, and, in fact, during packaging, distribution and storing until such time when the user has introduced the combination of doses into an inhaler and starts to inhale. Furthermore, the ratio between the active ingredients represents no problem, since it is a result of the metered dose quantities of the respective active ingredients constituting the combination of doses.

Although the medicament entities of the combination of doses are separated on the dose bed until the doses are to be delivered by a DPI, it is perfectly possible according to alternative embodiments of the invention to suck up the doses more or less mixed into the inspiration air during inhalation. In one aspect the powder entities of the combination of doses A′, B′ and optionally C′ may be sucked up simultaneously, partly or completely. The degree of intermixing of the delivered powders leaving the DPI mouthpiece may vary between 0 and 100% depending partly on the design of the DPI and its suction system, partly on the physical relative positions between deposited powder entities on the dose bed and partly on the relation between the dose bed and the suction system. For instance, if medicament A′ is deposited first onto a dose bed and B′ is then deposited on top of A′ and then C′ deposited on top of B′, the powders will be intermixed practically to 100% in the air when sucked up.

In another aspect the powder entities of a combination of doses may be sucked up sequentially, e.g. if the powder entities are accessed one at a time by the suction system of the DPI in the course of a single inhalation. Naturally, in that case, no mixing of powders will take place, since the delivery of the doses into inspiration air will be sequential, time separated.

In a third aspect, by selecting a pattern of physical positions and extensions in space of the deposited powder entities when forming the doses, it will be possible to tailor the prolonged delivery of the powders in the doses such that the medicament powders get intermixed into inspiration air to a selected degree between 0 and 100%.

Methods of dose forming include conventional mass or volumetric metering and devices and machine equipment well known to the pharmaceutical industry for filling blister packs, for example. See European Patent No. EP 0319131B1 and U.S. Pat. No. 5,187,921 for examples of prior art in volumetric and/or mass methods and devices for producing doses of medicaments in powder form. Electrostatic forming methods may also be used, for example as disclosed in U.S. Pat. Nos. 6,007,630 and 5,699,649.

Any suitable method capable of producing metered microgram and milligram quantities of dry powder medicaments may be used. Even completely different methods may be applied to suit the different medicaments selected to be part of the combination of doses to be produced. A dose may hold together in a more or less porous entity by action of van der Waals forces, electrostatic forces, electric forces, capillary forces etc interacting between particles and particle aggregates and the dose bed material.

Total mass in a combination of doses according to the present invention is typically in a range from 5 μg to 5 mg, but may extend to 50 mg. Regardless of which forming and filling method is being used for a particular medicament, it is important during dose forming to make sure that selected medicaments are individually metered and deposited onto their respective target areas or compartments of the dose bed. The target areas or compartments of the common dose bed, which aggregate to hold a particular combination of doses, may be of a same size or different sizes. The shape of compartments is governed by physical constraints defined by the type of dose bed used. As an example, a preferred type of dose bed is an elongated strip of a biologically acceptable, inert material, e.g. plastic or metal, between 5 and 50 mm long and between 1 and 10 mm wide. The strip may further be divided into separate target areas or compartments arranged along the length of the elongated strip. The dose bed or, if necessary each compartment receives an individual seal, for instance in the form of a foil, in a step immediately subsequent to the dose forming.

An advantage of the present invention is that the bronchodilators and steroid medicament are selected on merits of their own for inclusion in a combination of doses, in disregard of whether or not the respective formulations are compatible with one another. The medicament powders are kept separated in the manufacturing process and in the subsequent steps of distribution to the end user. Thus, the regulatory process before introducing a combination of doses of e.g. salbutamol sulphate, ipratropium bromide and fluticasone propionate on the market may be drastically simplified. Yet another advantage of the invention is the possibility of using pure, potent medicament substances for inclusion in the combination of doses, without any excipients.

TABLE 1
Typical dosages of beta2-agonists, anticholinergic agents and
corticosteroids currently on the market for treatment of respiratory
disorders

| | | Delivered dosage |
|---|---|---|
| Medicament active | Delivered dosage | range per day for |
| agent | range per dose (μg) | adults (μg) |
| Beta2-agonists | 1-100 | 1-200 |
| Anticholinergic agents | 1-400 | 1-2000 |
| Corticosteroids | 20-2000 | 20-5000 |

A combination of doses is intended for administration in a single inhalation, either irregularly when need arises, or more typically as part of a daily management regime. The number of combinations of doses administered regularly may vary considerably depending on the type of disorder, but typical dosing regimens are between one and four times daily. Optimal dosages and ratios of beta2-agonists, anticholinergic agents and corticosteroids respectively for prevention or treatment of respiratory disorders may be determined by those skilled in the art, and will vary with the respective potency of the selected medicaments and the advancement of the disease condition. Furthermore, factors associated with the individual undergoing treatment determine correct dosages, such as age, weight, sex etc. Depending on what constitutes correct dosages per day and the number of planned administrations per day, the correct deposits by mass for the selected medicaments may be calculated, such that metered deposits of each medicament entity to be included in the combination of metered doses may be produced in a dose-forming step.

Different dosing regimens may be chosen for different patients based on what treatment is considered best for an individual patient in her capacity as user of an inhaler device designed for a prolonged delivery of doses. In a first preferred embodiment each combination of doses comprises metered doses of each of the three medicaments A, B and optionally C in medicinally effective quantities and ratios. Each such combination of doses, i.e. A′, B′ and optionally C′, is made up of coordinated powder entities of the selected medicaments, A, B and optionally C, which are deposited onto a common dose bed and arranged for a prolonged delivery in a single inhalation by the user. The number of such administrations per day may range typically from one to four or five according to a prescribed dosing regimen.

In a second preferred embodiment, different dose combinations may be used in an inhaler device designed for a prolonged delivery of a combination of doses in a single inhalation. For instance, a combination of doses, A′+B′, comprising only two bronchodilating medicaments, A and B respectively, may be administered typically twice or three times a day, but interspersed with administration of a combination of doses, A′+B′+C′, comprising all three medicaments, A, B and C respectively, typically once or twice per day according to a selected, prescribed dosing regimen. The total quantities of each medicament thus administered per day and the total ratios between medicaments A, B and C in a day may of course be the same as in the first embodiment. Which embodiment is to be selected depends on which medicament quantities are required for an adequate treatment, preferences of the user, safety for the user and practical matters regarding forming of the dose combinations and restrictions concerning the number of dose beds per loading, which can be introduced into the inhaler of choice.

In a third preferred embodiment, similar to the second embodiment, a combination of doses, A′+B′, comprising only two bronchodilating medicaments, A and B respectively, may be administered typically from twice to four times a day using the inhaler for prolonged delivery. But administration of the combination of A′+B′ may be interspersed with administration of a dose C′ comprising only medicament C, typically once or twice per day using the same inhaler device according to a selected, prescribed dosing regimen.

In yet a fourth preferred embodiment, different dose combinations may be administered using two different inhalers, where the dose combinations and inhalers constitute a medicinal kit for use in the treatment of asthma, COPD and other respiratory disorders. The two inhalers are intended to be used in a co-ordinated administration of salbutamol (A) and ipratropium bromide (B), A and B together in a combined dose A′+B′, and (C) fluticasone propionate, in a separate dose C′. Such a kit may for instance comprise a metered dose inhaler (MDI) for combination of doses A′+B′ and a dry powder inhaler designed for a prolonged delivery for dose C′, or the kit may comprise two dry powder inhalers (DPI) designed for a prolonged delivery, one for combination A′+B′ and the other for dose C′. The metered dose inhaler Combivent® from Boehringer Ingelheim KG is an excellent example of an MDI, which is FDA-approved for combined doses A′+B′ of salbutamol and ipratropium bromide. The two inhalers are intended to be used in a co-ordinated administration of combined doses A′+B′, which may be administered several times a day, and dose C′, normally administered once daily, although in separate inhalations. An objective of the fourth embodiment is to combine the very successful use of Combivent® or an equivalent inhaler in as-needed administration of bronchodilators with an effective administration of low doses of fluticasone to treat an inflammation of the bronchi, but without adverse side effects, or nearly so, by implementing the use of a dry powder inhaler for a prolonged, more effective delivery of a fluticasone powder dose.

In calculating a correct nominal deposit of mass for each medicament the fine particle fraction, i.e. particles having a mass median aerodynamic diameter (MMAD) less than 5 μm, of the actual delivered dose per medicament must be taken into account. As discussed in the foregoing, the efficacy of inhalers differs considerably and it is thus important to include the expected efficacy of the chosen inhaler in the calculation of a suitable nominal mass in the deposited entity or entities of each dose. What constitutes suitable amounts of the three medicaments A, B and optionally C and the respective optimal masses of bronchodilators and steroid respectively depend on the factors described in the foregoing. Generally the inhaled beta2-agonist mass, e.g. in the form of salbutamol, per dose should be in a range from 1 to 100 μg, preferably 2 to 50 μg and inhaled anticholinergic agent, e.g. in the form of ipratropium bromide, per dose should be between 1 and 400 μg and a corticosteroid, e.g. fluticasone propionate per dose in a range from 20 to 2000 μg, preferably between 40 and 1000 μg.

There is generally a medical need to direct the delivery, i.e. the deposition, of inhaled doses of a medicament to the optimum site of action in the lung, where the therapeutic effect is the best possible, either for a topical effect or for a systemic effect. Turning to the case in point, it is of course desirable to control the deposition of the combination of doses of bronchodilators and steroid to their respective sites of action in the lung in order to get highest possible overall efficacy for each dose with a minimum of adverse side effects. Aerodynamic particle size is a most important factor greatly influencing where in the lung particle deposition is likely to take place. From a target site point of view, it is therefore desirable to tailor the physical formulations of the respective medication powders in the combination of doses in such a way that they result in an advantageous particle aerodynamic size distribution by mass in the delivered dose. The present invention makes it possible to deliver the combination of doses, thus formulated, to the targeted sites of action. Available data indicate that for best performance, the AD (aerodynamic diameter) for a steroid as delivered should be in a range from 2 to 8 μm for a more central lung deposition, whereas AD for beta2-agonists and anticholinergic agents in the delivered dose should be in a range from 1 to 5 μm for a deposition in the more peripheral lung.

Another circumstance to consider is the order of delivery for the combination of doses of the present invention. The first air to be sucked in by a person inhaling reaches deep into the peripheral lung and air sucked in thereafter fins up the lungs gradually. What this means is that powders intended for a peripheral lung deposition should be inhaled early in the inhalation cycle to maximize deposition in that area and powders intended for a central lung deposition should be inhaled somewhat later in the cycle to maximize deposition in the central lung. Since available data suggest that a beta2-agonist and an anticholinergic agent should preferably deposit in the peripheral lung area and the steroid in the central lung area, a dose of beta2-agonist with the fastest onset should be the first to be sucked in followed by a dose of anticholinergic agent and last the steroid. Under the proviso that an adapted DPI is at hand for a prolonged, sequential delivery of the combination of doses in the course of a single inhalation, the present invention refutes prior art and claims that sequential delivery of a combination of doses is to be preferred compared to simultaneous delivery, e.g. combined doses in the form of a mixture. Compared to prior art the present invention presents a definite advantage regarding high delivered dose efficacy and benefits for the user, including negligible adverse side effects.

The present invention makes use of proven dry powder formulations of bronchodilators and steroids, i.e. a beta2-agonist A, an anticholinergic agent B and a steroid C, where A is represented by salbutamol, salmeterol or formoterol and B is represented by oxitropium, or preferably ipratropium nor more preferably tiotropium, all of which advantageously in bromide form and C is represented by budesonide or more preferably fluticasone, particularly fluticasone propionate, finely divided and adapted for separate deposition onto a common dose bed, normally with no mixing of the powders A, B and C. A combination of doses thus formed, i.e. A′, B′ and optionally C′, may be introduced into an adapted dry powder inhaler (DPI) such that the medicament entities constituting the combination of doses may be aerosolized and delivered in the inspiration air during the course of a single inhalation through a DPI by a user. Keeping the different deposited medicament entities separated according to the invention may reduce the investment in time and resource necessary for getting the combination of doses approved by the relevant regulatory bodies and released to the respective markets. For instance, no added substance to stabilize the combination of doses will be needed and no testing to prove that an added substance is harmless needs to be performed.

The present invention differs from prior art inhalers and related combined dose delivery methods by providing combination of doses comprising two or optionally three coordinated, individually proven asthma medicaments in form of separately deposited entities onto a common dose bed. The combination of doses is therefore not a single composition of asthma medicaments constituting a single physical entity. The invention discloses combinations of doses comprising at least one physical entity per medicament loaded and coordinated onto a common dose bed with an objective of providing more efficient dose delivery and treatment of asthma. Inserted into a DPI, designed for a prolonged delivery, the combination of doses will be aerosolized during a single inhalation by a user. Preferably, the entities of a combination of doses of bronchodilators and anti-inflammatory steroid will be delivered sequentially or optionally more or less simultaneously into the inspiration air. Whether a combination of doses of medicaments is aerosolized sequentially or simultaneously depends on the physical form of the combination of doses, i.e. how the deposited medicament entities are interrelated, and on the type of inhaler used to administer the combination of doses.

It is obvious that an inhaler, which instantaneously subjects all powders of the combination of doses to a jet-stream of air will aerosolize the aggregated entities more or less simultaneously, whereby the medicament powders, still more or less agglomerated, become mixed into the air leaving the mouthpiece. In contrast, an inhaler subjecting the combination of doses to a jet stream gradually, like a moving tornado attacking adjacent corn fields, one after the other, thereby not attacking all of the powder entities of the combination of doses instantly, may aerosolize the entities of the combination of doses gradually over time. An object of the invention is to offer better control of dose release and to facilitate a prolonging of dose delivery in order to produce a high fine particle fraction (FPF) in the delivered doses of the combination. Another object of the invention is to achieve a high ratio of delivered relative metered dose mass of each of the medicament components of the combination. Although it is possible to successfully apply the invention to prior art inhalers, they tend to deliver the combination of doses more or less mixed in too short a time, resulting in a poor FPF figure and low efficacy. On the other hand, a gradual, well-timed, sequential delivery of the combination of doses is possible using a new inhaler design where a relative movement is introduced between the combination of doses and a suction nozzle through which the inspiration airflow is channeled. This arrangement utilizes the inhalation effort of the user to aerosolize the combination of doses gradually for a prolonged period, thus using the power of the suction more efficiently and eliminating in most cases a need for external power to aerosolize the combination of doses. A novel device for aerosolizing a dry powder dose is disclosed in our US Application No. US 2003/0192538 A1 and a method of de-aggregating and dispersing dry medicament powder into air is disclosed in our US Application No. US 2003/0192539 A1. Both documents are hereby incorporated in this document in their entirety as references.

A powder Air-razor method is advantageously used for aerosolizing the medicament powder entities of the combination of doses, the Air-razor providing de-aggregation and dispersal into air of the finely divided medication powders. By utilizing an effort of sucking air through a mouthpiece of an inhaler, said mouthpiece connected to a nozzle, the particles of the deposited medicament powders, made available to the nozzle inlet, are gradually de-aggregated and dispersed into a stream of air entering the nozzle. The gradual de-aggregation and dispersal is produced by the high shearing forces of the streaming air in connection with a relative motion introduced between the nozzle and the powder entities of the combination of doses. In a preferred embodiment, the medicament powders are deposited onto a dose bed, such that the powder deposits occupy an area of similar or larger size than the area of the nozzle inlet. The nozzle is preferably positioned outside the area of deposits, not accessing the powder by the relative motion until the air stream into the nozzle, created by an applied suction, has passed a threshold flow velocity. Coincidental with the application of the suction or shortly afterwards the relative motion will begin such that the nozzle traverses the powder entities constituting the combination of doses gradually. The high velocity air going into the nozzle inlet provides plenty of shearing stress and inertia energy as the flowing air hits the leading point of the border of the contour of the medicament entities, one after the other. This powder Air-razor method, created by the shearing stress and inertia of the air stream, is so powerful that the particles in the particle aggregates in the powder adjacent to the inlet of the moving nozzle are released, de-aggregated to a very high degree as well as dispersed and subsequently entrained in the created air stream going through the nozzle. If the medicament deposits have been made in separate compartments of the dose bed and individually sealed, then obviously the compartments must be opened up first so that the nozzle can access the deposited powder entities in each compartment when suction is applied. Naturally, this is also true if the deposits share a common seal without an individual seal for each deposited entity. An arrangement for cutting foil is disclosed in our Swedish patent publication SE 517 227 C2 (WO 02/24266 A1), which is hereby incorporated in this document in its entirety as a reference. Depending on how the entities are laid out on the dose bed, the nozzle will either suck up the powder entities sequentially or in parallel or in some serial/parallel combination.

The present invention improves the efficacy of dose delivery, compared to the best selling inhalers on the market today, by at least a factor of two and typically 2.5. This is accomplished by raising the FPF <5 μm in the delivered dose to more than 40%, preferably to more than 50%, by mass, compared to typically less than 30% for prior art inhalers. The implications of this vast improvement are much less adverse reactions in users, even to the point of eliminating the risk of death, due to high dosages of beta2-agonists, anticholinergic agents or corticosteroids systemically or in the wrong parts of the lung.

Thus, the quality of asthma medicament delivery is dramatically improved compared to prior art performance, leading to important advances in delivering a majority of fine particles of the asthma medicaments of the combination of doses to the intended target area or areas in the user's airways and lungs with very little loss of particles settling in the throat and upper airways. Administering asthma medicament combinations according to the present invention has a very positive therapeutic effect from a medical, psychological and social point of view on a user in need of asthma treatment with a co-ordinated combination of a beta2-agonist, an anticholinergic agent and an anti-inflammatory steroid.

### DETAILED DESCRIPTIONS OF DRAWINGS

Referring to reference numbers 1-100 of the drawings wherein like numbers indicate like elements throughout the several views of ten different embodiments of a combination of doses comprising at least two deposited entities of two medicaments onto a dose bed as illustrated in FIGS. 1-10 presented here as non-limiting examples.

FIG. 1 illustrates a combination of doses 100 comprising two different medicament entities deposited, 1 and 2, in separate compartments 21 and 22 onto a dose bed 20, said compartments may be capsules or blisters or moldings in the dose bed. An individual seal 13 for each compartment guarantees that the medicament doses cannot be contaminated by foreign matter or by one another. The illustrated doses are intended for a sequential delivery taking place during a single inhalation.

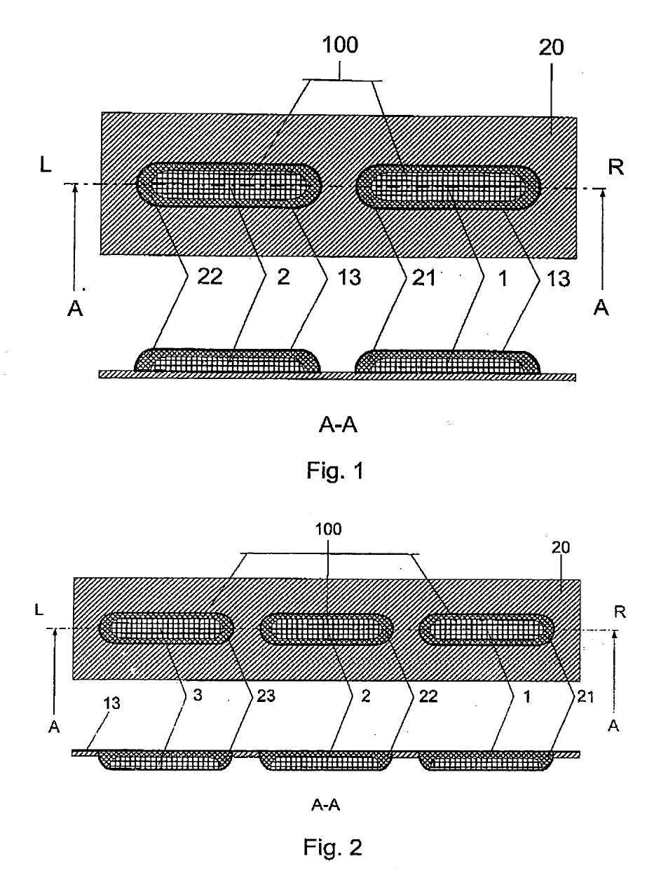

FIG. 2 illustrates a combination of doses 100 comprising three different medicament entities, 1, 2 and 3 in separate compartments 21, 22 and 23 similar to FIG. 1, but arranged underneath the dose bed 20. Besides a different arrangement of compartments on the dose bed 20 and the respective seals 13, the main difference between FIG. 1 and FIG. 2 is that entity 3 consists of the medicament of entity 2. It is thus possible not only to administer two medicaments, but also to compose the combination of doses of two medicaments with a very high ratio of mass between them. The illustrated deposited entities are intended for a sequential delivery taking place during a single inhalation.

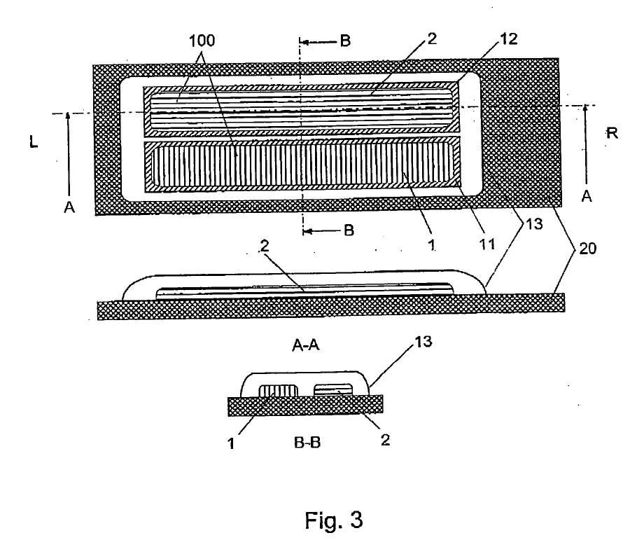

FIG. 3 illustrates a combination of doses 103 comprising two different medicament entities, 1 and 2, laid out in parallel strips onto separate target areas 11 and 12 respectively onto the dose bed 20. A common protective foil 13 protects the medicaments of the a combination of doses from being contaminated by foreign matters. The illustrated entities are intended for a fully simultaneous delivery of the two medicaments taking place during a single inhalation.

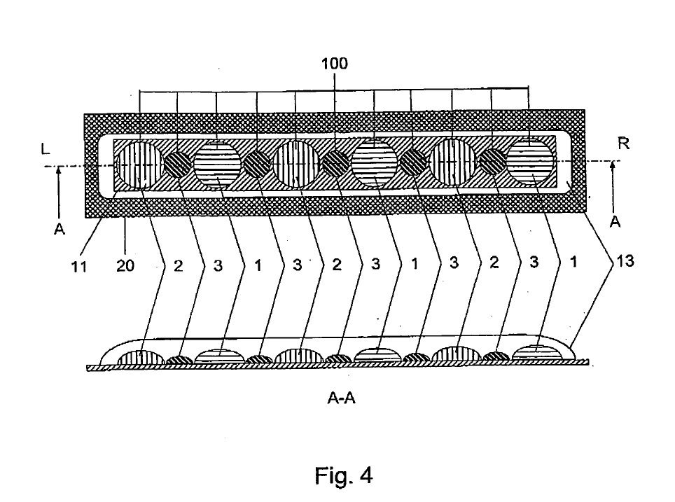

FIG. 4 illustrates a combination of doses 100 comprising two different medicaments, 1 and 2, each comprising several deposited entities separated by deposited entities of an inert excipient 3. The deposited entities are laid out in a string of spots onto a target area 11 on a dose bed 20. The entities share a common seal 13. The combination of doses is intended for a sequential delivery of incorporated medicament and excipient entities, said delivery taking place during an inhalation. The excipient deposits help to minimize unintentional mixing of the medicaments. If some mixing of medicaments can be accepted, then the excipient entities may be left out altogether. A combination of doses composed of spot entities may of course comprise more medicaments than two. The mass ratio between medicament doses may be easily set by controlling the ratio between the number of spot entities per medicament in combination with the size of the respective spot entities in terms of deposited mass. Naturally the spot entities need not necessarily be circular in shape, they may take an elongated or elliptical form, depending on which types of combined dose forming methods are used.

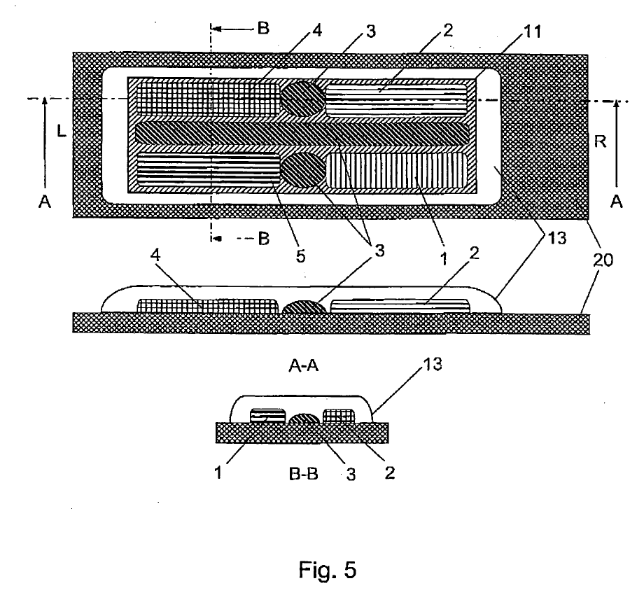

FIG. 5 illustrates a combination of doses 100 comprising deposited entities representing up to four different medicaments, 1, 2, 4 and 5, separated by deposited entities of an inert excipient 3. The deposited entities are laid out in two parallel groups of two entities per group lined up in strips onto a common target area 11 on a dose bed 20. The deposited entities share a common seal 13. The excipient deposited entities help to minimize unintentional interaction of the medicament doses. The combination of doses is intended for a combined parallel/simultaneous and sequential delivery of incorporated medicament doses, said delivery taking place during a single inhalation.

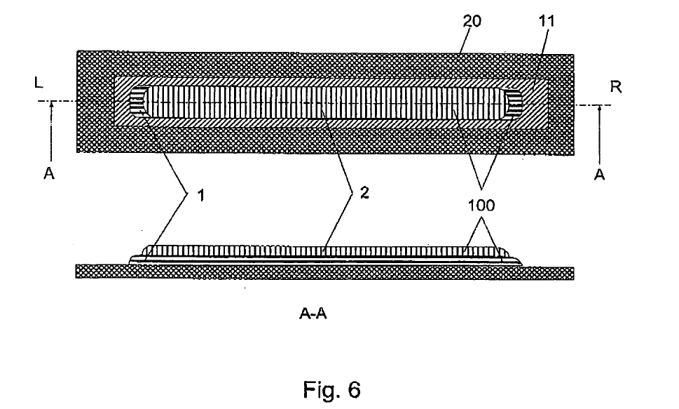

FIG. 6 illustrates a combination of doses 100 comprising two different medicament entities, 1 and 2, each comprising a strip of deposited powder, medicament 1 deposited onto a target area 11 of a dose bed 20 and medicament 2 deposited on top of the entity of medicament 1. This method of forming a combination of doses is an alternative to the ones previously disclosed and may be used when a certain level of interaction or mixing of the medicaments may be tolerated.

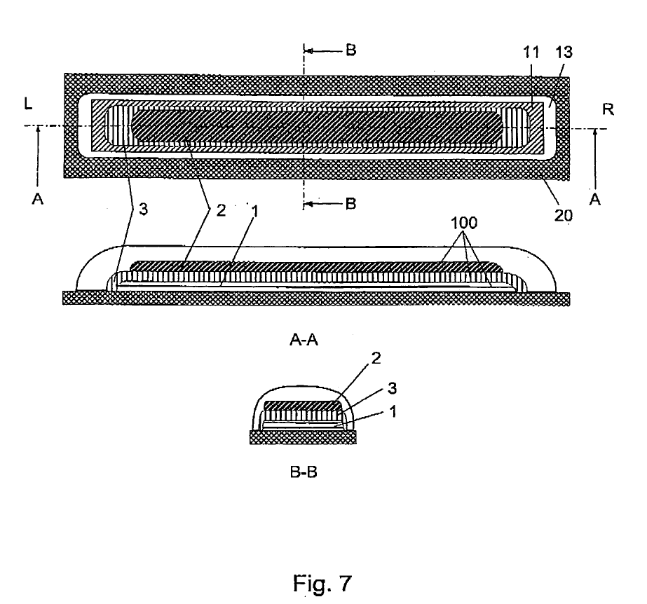

FIG. 7 illustrates a combination of doses 100 comprising two different medicament entities, 1 and 2, and an excipient entity 3, each comprising a strip of deposited powder. Medicament 1 is deposited onto a target area 11 of a dose bed 20 and excipient 3 is deposited onto medicament 1 to insulate medicament 1 from a deposit of medicament 2 on top of the deposited entity of excipient 3.

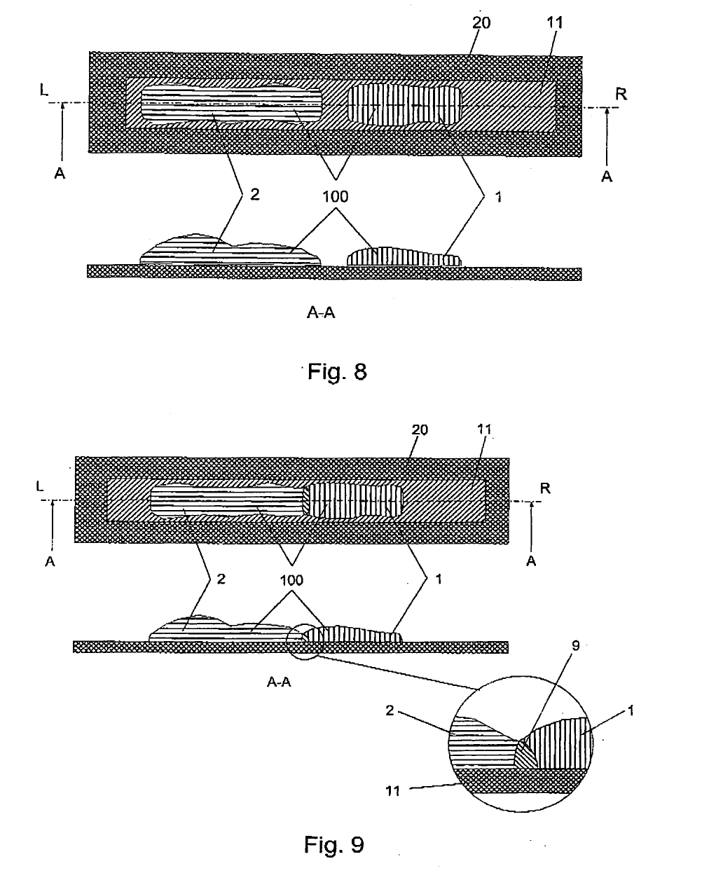

FIG. 8 illustrates a combination of doses 100 comprising two different medicament entities, 1 and 2, of somewhat irregular shapes but separately laid out onto a common target area 11 of the dose bed 20. The illustrated entities are intended for a sequential delivery of the two medicament doses taking place during an inhalation.

FIG. 9 illustrates a combination of doses 100 comprising two different medicament entities, 1 and 2, of somewhat irregular shapes but generally separately laid out onto a common target area 11 of the dose bed 20. The illustrated deposited entities overlap slightly, resulting in an arbitrary mixture 9. The deposits are intended for a mostly sequential delivery of the two medicament doses taking place during a single inhalation.

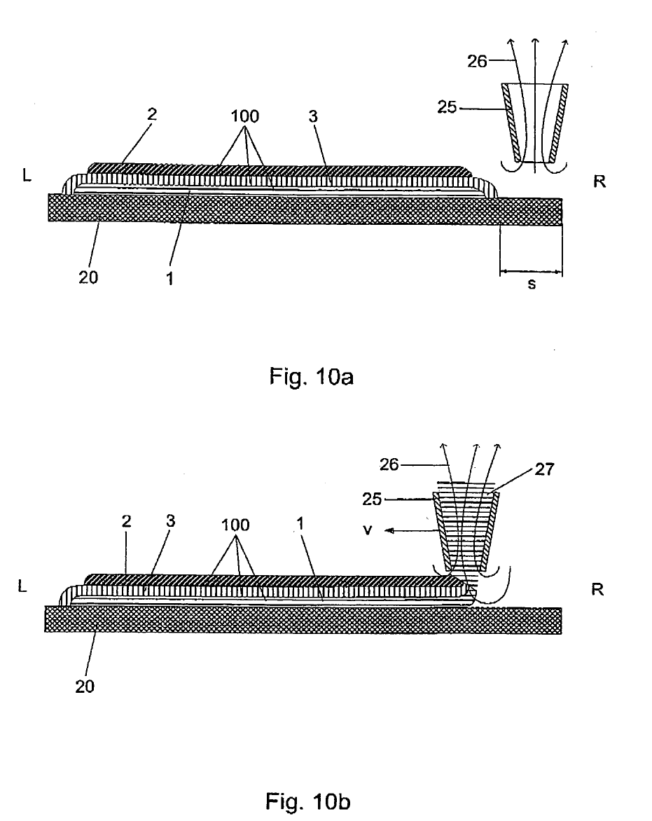

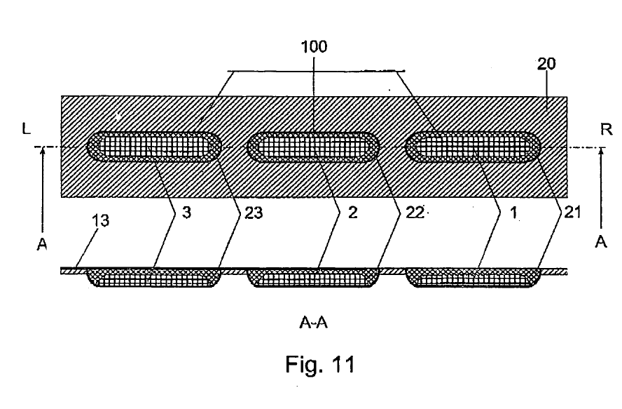

FIGS. 10a and 10b illustrate a delivery of a combination of doses 100 comprising two different medicament entities, 1 and 2, and an excipient entity 3, each comprising a strip of powder sequentially deposited in three different layers. A nozzle 25 with an established flow of air 26 going into the inlet is put in a relative motion, parallel to the dose bed 20, such that the nozzle passes over the a combination of doses beginning at the right side R and ending at the left side L of the dose bed. This Air-razor method results in a simultaneous, gradual delivery of medicament entities 1 and 2 together with the excipient entity 3. The powders of the entities are mixed into an aerosol 27 by the air flowing into the nozzle leading to simultaneous delivery of the two medicament doses and the excipient. This Air-razor method may be applied to all embodiments of the present invention and results in a simultaneous or sequential or a combined simultaneous/sequential delivery of all included medicament doses and optional excipients.

FIG. 11 illustrates a combination of doses 100 comprising three different medicaments, 1, 2 and 3 in separate compartments 21, 22 and 23 similar to FIG. 1, but arranged underneath the dose bed 20. Besides a different arrangement of compartments on the dose bed 20 and the respective seals 13, the main difference between FIG. 1 and FIG. 11 is that entity 3 consists of a third medicament. It is thus possible to administer a combination of three medicament doses. The illustrated deposited entities are intended for a sequential delivery during a single inhalation.

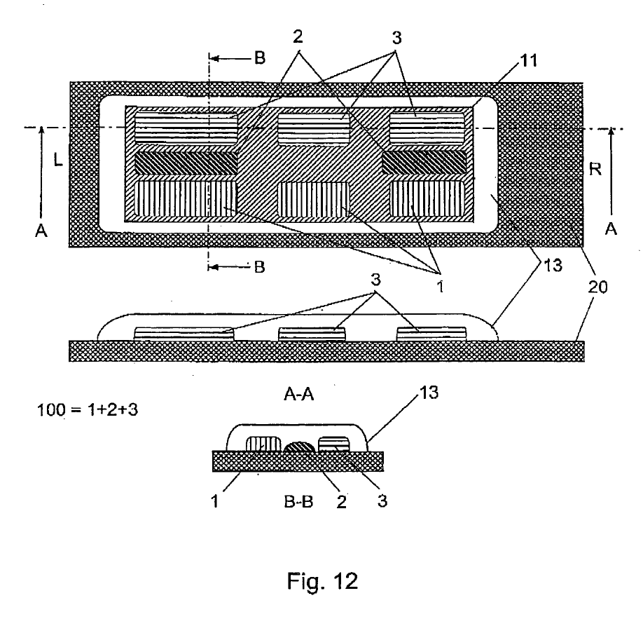

FIG. 12 illustrates a combination of doses 100 comprising deposited entities representing three different medicaments, 1, 2, and 3, each constituted of three separately deposited entities, except medicament 2, which has only two entities. The deposited entities are laid out in three parallell groups of two and three entities per group lined up in elongated dots onto a target area 11 on a common dose bed 20. The deposited entities share a common seal 13. The combination of doses is intended for a mixed parallel/simultaneous and sequential delivery of incorporated medicament doses, said delivery taking place during a single inhalation.

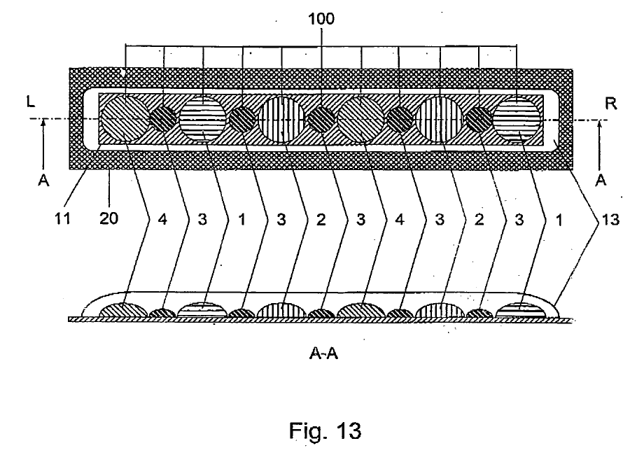

FIG. 13 illustrates a combination of doses 100 comprising three different medicaments, 1, 2 and 4 each comprising several deposited entities separated by deposited entities of an inert excipient 3. The deposited entities are laid out in a string of spots onto a target area 11 on a common dose bed 20. The entities share a common seal 13. The combination of doses is intended for a sequential delivery of incorporated medicament and excipient entities, said delivery taking place during an inhalation. The excipient deposits help to minimize unintentional mixing of the medicaments. If some mining of medicaments can be accepted; then the excipient entities may be left out altogether. The mass ratio between medicament doses may be easily set by controlling the ratio between the number of spot entities per medicament in combination with the size of the respective spot entities in terms of deposited mass. Naturally the spot entities need not necessarily be circular in shape, they may take an elongated or elliptical form, depending on which dose forming methods are used.

1. A method for the administration of a combination of metered doses of finely divided dry medication powders using a dry powder inhaler device, comprising the steps of
selecting medicaments A, B and C for a forming of a pharmaceutical combination of doses, A′, B′ and C′ respectively, where A stands for a beta2-agonist or a pharmaceutically acceptable salt, enantiomer, racemate, hydrate, or solvate including mixtures thereof, and B stands for an anticholinergic agent or a pharmaceutically acceptable salt, enantiomer, racemate, hydrate, or solvate including mixtures thereof, and C stands for an anti-inflammatory corticosteroid or a pharmaceutically acceptable salt enantiomer, racemate, hydrate, or solvate including mixtures thereof, and where A, B and C may optionally further include excipients;
preparing the medicinal combination of metered doses, A′, B′ and C′, comprising separately metered deposits of medicinally effective quantities of each of the medicaments A, B and C onto a common dose bed, the sum of the deposits constituting the metered quantities of powder of the medicinal combination of doses;
introducing the combination of doses into an inhaler device adapted for a prolonged dose delivery, and when suction is applied through the inhaler, the powders of each of the doses A′, B′ and C′ are aerosolized, generally presenting a fine particle fraction, FPF, of at least 30-50%, of delivered powder mass, whereby the combined doses A′, B′ and C′ are delivered either simultaneously or separately in sequence, or in a combination thereof.

2. The method according to claim 1, comprising the further step of
aerosolizing the deposited powders of the combined doses gradually over a time-period during an inhalation through the inhaler device.

3. The method according to claim 1, comprising the further step of
selecting as medicament A one of the beta2-agonists salbutamol sulphate or formoterol fumarate or salmeterol xinafoate and as medicament B one of the anticholinergic agents ipratropium bromide or tiotropium bromide or oxitropium bromide and as medicament C one of the corticosteroids fluticasone propionate or budesonide, each medicament optionally including excipients, in forming the combination of doses.

4. The method according to claim 1, comprising the further step of
arranging each combination of doses such that when a combination of doses is introduced for inhalation in the inhaler, the metered doses of medicaments A, B and optionally C are sucked up sequentially in order, starting with a dose of medicament A, followed by a dose of medicament B and optionally followed last by a dose of medicament C, whereby the medicament powder doses consisting of medicaments A, B and optionally C respectively will be separately delivered and deposited.

5. The method according to claim 1, comprising the further step of
arranging each combination of doses such that when a combination of doses is introduced for inhalation in the inhaler, the metered doses of medicaments A, B and optionally C are sucked up together, the medication powders of A, B and optionally C thus delivered as a mixed aerosol.

6. The method according to claim 1, comprising the further step of
preparing the dry powder medicinal combination of doses to a total mass in a range from 5 μg to 50 mg.

7. The method according to claim 1, comprising the further step of
separating deposited entities of the included medicinal drugs from each other onto a dose bed, such that the medicaments cannot detrimentally mix with each other after forming of the combination of doses.

8. The method according to claim 1, comprising the farther step of
selecting the inhaler device to be an adapted dry powder inhaler (DPI) designed for a prolonged delivery of medicinal combinations of doses, such that the inhaler device will administer only one combination of doses at a given instant.

9. A method for the administration by inhalation of a combination of metered doses of finely divided dry medication powders using a dry powder inhaler device, comprising the steps of
selecting medicaments A and B for a forming of a pharmaceutical combination X of doses A′ and B′ respectively, where A stands for a beta2-agonist or a pharmaceutically acceptable salt, enantiomer, racemate, hydrate, or solvate including mixtures thereof, and B stands for an anticholinergic agent or a pharmaceutically acceptable salt, enantiomer, racemate, hydrate, or solvate including mixtures thereof, and where A and B may optionally further include excipients;
further selecting medicaments A, B and C for a forming of a pharmaceutical combination Y of doses A″, B″ and C″ respectively, where A stands for a beta2-agonist or a pharmaceutically acceptable salt, enantiomer, racemate, hydrate, or solvate including mixtures thereof, and B stands for an anticholinergic agent or a pharmaceutically acceptable salt, enantiomer, racemate, hydrate, or solvate including mixtures thereof, and C stands for an anti-inflammatory corticosteroid or a pharmaceutically acceptable salt, enantiomer, racemate, hydrate, or solvate including mixtures thereof, and where A, B and C may optionally further include excipients;
preparing the medicinal combinations, X and Y respectively; of metered doses, each combination comprising separately deposited entities of medicinally effective quantities of each of the selected medicaments onto a common dose bed, one for combination X and another one for combination Y, the sum of the deposited entities per dose bed constituting the metered doses of powder of the respective medicinal combination, X and Y, of doses;
introducing the combinations X and Y into an inhaler device adapted for a prolonged dose delivery, and when suction is applied through the inhaler, the powders of each of the entities of a selected dose combination are aerosolized, generally presenting a fine particle fraction, FPF, of at least 30-50% of delivered powder mass, whereby the entities of the combination of doses are delivered either simultaneously or separately in sequence, or in a combination thereof, and
organizing the sequence of combinations X and Y to fit a desired dosing regimen for the inhaler device.

10. The method according to claim 9, comprising the further step of
aerosolizing the deposited powders of the combined doses gradually over a time-period during an inhalation through the inhaler device.

11. The method according to claim 9, comprising the further step of
picking out medicament A from a group of beta2-agonists comprising salbutamol sulphate, formoterol fumarate and salmeterol xinafoate and picking out medicament B from a group of anticholinergic agents comprising ipratropium bromide, tiotropium bromide and oxitropium bromide,
further picking out medicament A from a group of beta2-agonists comprising salbutamol sulphate, formoterol fumarate and salmeterol xinafoate and picking out medicament B from a group of anticholinergic agents comprising ipratropium bromide, tiotropium bromide and oxitropium bromide and picking out medicament C from a group of corticosteroids comprising fluticasone propionate and budesonide.

12. The method according to claim 9, comprising the further step of
arranging each combination of doses such that when a combination of doses is introduced for inhalation in the inhaler, the metered doses of medicaments A, B and optionally C are sucked up sequentially in order, starting with a dose of medicament A, followed by a dose of medicament B and optionally followed last by a dose of medicament C, whereby the medicament powder doses consisting of medicaments A, B and optionally C respectively will be separately delivered and deposited.

13. The method according to claim 9, comprising the further step of
arranging each combination of doses such that when a combination of doses is introduced for inhalation in the inhaler, the metered doses of medicaments A, B and optionally C are sucked up together, the medication powders of A, B and optionally C thus delivered as a mixed aerosol.

14. The method according to claim 9, comprising the further step of
preparing the dry powder medicinal combination of doses to a total mass in a range from 5 μg to 50 mg.

15. The method according to claim 9, comprising the further step of
separating deposited entities of the included medicinal drags from each other onto a dose bed, such that the medicaments cannot detrimentally mix with each other after forming of the combination of doses.

16. The method according to claim 9, comprising the further step of selecting the inhaler device to be an adapted dry powder inhaler (DPI) designed for a prolonged delivery of medicinal combinations of doses, such that the inhaler device will administer only one combination of doses at a given instant.

17. A combination of doses of pharmaceutical dry powders, adapted for administration by inhalation using a dry powder inhaler device (DPI), said inhaler device designed for a prolonged delivery of the combination of doses wherein
medicaments A, B and optionally C are selected for a forming of a pharmaceutical combination of doses, A′, B′ and optionally C′ respectively, where A stands for a beta2-agonist or a pharmaceutically acceptable salt, enantiomer, racemate, hydrate, or solvate including mixtures thereof, and B stands for an anticholinergic agent or a pharmaceutically acceptable salt, enantiomer, racemate, hydrate, or solvate including mixtures thereof, and C stands for an anti-inflammatory corticosteroid or a pharmaceutically acceptable salt, enantiomer, racemate, hydrate, or solvate including mixtures thereof, and where A, B and C may optionally further include excipients;
the combination of doses of pharmaceutical dry powders are prepared to comprise separate, metered entities of medicinally effective quantities of the selected medicaments respectively, where the sum of the deposited entities constitutes the metered quantities of powder in the pharmaceutical combination of doses;
the entities of the combination of doses are co-ordinated during preparation such that, after having been introduced into an adapted inhaler, when suction through the inhaler is applied, the powders of the entities are aerosolized, whereby the entities of the combination of doses are delivered to and deposited in the airways either simultaneously or separately in sequence, or in some combination thereof, during a single inhalation effort by user.

18. The combination of doses according to claim 17, wherein
medicament A is selected as one of the beta2-agonists salbutamol sulphate or formoterol fumarate or salmeterol xinafoate and medicament B is selected as one of the anticholinergic agents ipratropium bromide or tiotropium bromide or oxitropium bromide and optionally medicament C is selected as one of the corticosteroids fluticasone propionate or budesonide, each medicament optionally including excipients, in forming the combination of doses.

19. The combination of doses according to claim 17, wherein
each combination of doses is arranged such that when a combination of doses is introduced for inhalation in the inhaler, the metered doses of medicaments A, B and optionally C are sucked up sequentially in order, starting with a dose of medicament A, followed by a dose of medicament B and optionally followed by a dose of medicament C, whereby the medicament powder doses consisting of medicaments A, B and optionally C respectively will be separately delivered and deposited.

20. The combination of doses according to claim 17, wherein
each combination of doses is arranged such that when a combination of doses is introduced for inhalation in the inhaler, the metered doses of medicaments A, B and optionally C are sucked up together, the medication powders of A, B and optionally C thus delivered as a mixed aerosol.

21. The combination of doses according to claim 17, wherein
the dry powder medicinal combination of doses is prepared to a total mass in a range from 5 μg to 50 mg.

22. The combination of doses according to claim 17, wherein
deposited entities of the included medicaments are separated from each other onto a dose bed, such that the medicaments cannot detrimentally mix with each other after forming of the combination of doses.

23. The combination of doses according to claim 17, wherein
the inhaler device is chosen to be an adapted dry powder inhaler designed for a prolonged delivery of medicinal combinations of doses, such that one inhalation will administer only one combination of doses at a time.

24. A use of differently acting dry powder medicaments, intended to be combined in an inhaler device, wherein
medicaments A, B and optionally C are selected for a forming of a pharmaceutical combination of doses, A′, B′ and optionally C′ respectively, where A stands for a beta2-agonist or a pharmaceutically acceptable salt, enantiomer, racemate, hydrate, or solvate including mixtures thereof, and B stands for an anticholinergic agent or a pharmaceutically acceptable salt, enantiomer, racemate, hydrate, or solvate including mixtures thereof, and C stands for an anti-inflammatory corticosteroid or a pharmaceutically acceptable salt, enantiomer, racemate, hydrate, or solvate including mixtures thereof, and where A, B and C may optionally further include excipients;
a suitable pattern is selected of physical positions and extensions in space for separate depositions onto a common dose bed of metered powder entities constituting the combination of doses;
separate, metered powder entities of selected medicaments are deposited in the suitable pattern onto the common dose bed and
the entities of the combination of doses are co-ordinated during preparation such that, after having been introduced into an inhaler device adapted for a prolonged delivery, the entities of the different medicament powders, A′, B′ and C′, when sucked up become aerosolized and delivered to and deposited separately in a timed sequence, therefore generally without a mixing.

25. A medical kit comprising combinations of doses of asthma medicaments, adapted for administration by inhalation using two inhaler devices, wherein
medicaments A and B are selected for a forming of a pharmaceutical combination of doses, A′+B′, where A stands for a beta2-agonist dose or a pharmaceutically acceptable salt enantiomer, racemate, hydrate, or solvate including mixtures thereof, and B stands for an anticholinergic agent or a pharmaceutically acceptable salt, enantiomer, racemate, hydrate, or solvate including mixtures thereof and where the combination may optionally further include excipients;
a medicament C is further selected for a forming of a pharmaceutical dry powder dose C′, where C stands for an anti-inflammatory corticosteroid or a pharmaceutically acceptable salt, enantiomer, racemate, hydrate, or solvate including mixtures thereof, optionally further including excipients;
the pharmaceutical combination of doses A′+B′ is introduced into a selected inhaler device;
the pharmaceutical dry powder dose C′ is introduced in a dry powder inhaler device (DPI) adapted for a prolonged dose delivery;
a user administers the combination A′+B′ by operating the selected inhaler device, optionally followed by operating the DPI to also administer a dose C′ in accordance with a desired dosing regimen, and
when being administered the powder of dose C′ is gradually aerosolized by the DPI, generally presenting a fine particle fraction, FPF, of at least 30-50% in the powder mass of the delivered dose, whereby most of dose C′ is delivered to and deposited in the desired site.

26. The medical kit according to claim 25, further characterized in that
the selected inhaler device is a Combivent® MDI inhaler from Boehringer Ingelheim KG.

27. The medical kit according to claim 25, further characterized in that
the selected inhaler device is a dry powder inhaler device adapted for a prolonged dose delivery.
